(19) United States
(12) Patent Application Publication
(10) Pub. No.: US 2018/0130554 A1
(43) Pub. Date: May 10, 2018

(54) PATIENT FLOW TIMER SOFTWARE

(71) Applicant: Jack Cheng, Buford, GA (US)

(72) Inventor: Jack Cheng, Buford, GA (US)

(21) Appl. No.: 15/624,690

(22) Filed: Jun. 15, 2017

Related U.S. Application Data
(60) Provisional application No. 62/350,678, filed on Jun. 15, 2016.

(51) Int. Cl. G16H 40/20; G06Q 10/06

(52) U.S. Cl. CPC G16H 40/20; G06Q 10/0633

(57) ABSTRACT

Implementations of a Patient Flow Timer (PFT) system for measuring a patient's movement and wait times through a medical office (i.e., patient flow) are provided. In some implementations, for each activity, office person to be seen, or event that the patient will encounter during the patient's office visit, a start time, stop time, and total time spent may be recorded using the PFT system. In some implementations, the wait time between adjacent activities, office persons, or events, may be calculated and recorded. The data may be captured using a computing device having a user-interface for capturing start times and stop times. By capturing data real-time, real-time displays can be created to show the state of patient flow in an office and office wait times can be computed.

### CROSS REFERENCE TO RELATED APPLICATION

This application claims the benefit of U.S. Patent Application Ser. No. 62/350,678, which was filed on Jun. 15, 2016, and is incorporated herein by reference in its entirety.

### TECHNICAL FIELD

This disclosure relates to implementations of a Patient Flow Timer (PFT) system.

### BACKGROUND

Existing systems, such as the system disclosed in U.S. Pat. No. 7,925,603, for measuring patient flow are location-based. That is, existing systems may measure times that a patient moves from one location to another location. These existing systems do not measure the wait times between activities. For example, a plurality of activities may occur at one location and existing systems cannot measure wait times between activities. Existing systems, such as the system disclosed in U.S. Patent Appl. Pub. No. 2012/0226508, also do not measure end times for various activities in a patient flow.

### BRIEF DESCRIPTION OF THE DRAWINGS

FIG. 1A illustrates example raw data captured using the Patient Flow Timer (PFT) system according to an implementation of the present disclosure.

FIGS. 1B and 2-4 illustrates sample reports that may be generated from the data gathered using the PFT system according to implementations of the present disclosure.

FIG. 5 illustrates an example user interface to allow office personnel to easily record interaction times with a patient according to an implementation of the present disclosure.

FIG. 6 illustrates an example real time display of a physician's schedule with the actual progression of the schedule immediately underneath it based on data gathered using the PFT system according to an implementation of the present disclosure.

FIG. 7 illustrates an example environment 700 for recording interaction times with a patient according to an implementation of the present disclosure.

FIG. 8 illustrates another example environment 800 for recording interaction times with a patient using the PFT system according to an implementation of the present disclosure.

FIG. 9 illustrates an example computer system 900, which may be used with some embodiments of the present invention.

### DETAILED DESCRIPTION

Implementations of a Patient Flow Timer (PFT) system for measuring a patient's movement and wait times through a medical office (i.e., patient flow) are provided.

In some implementations, for a plurality of activities at a medical office for a patient as the patient moves through a medical office for services, the PFT system receives for each activity for each patient at a first computing device an actual start time of the activity and an actual stop time of the activity. In some implementations, the actual start times and actual stop times are transmitted over a network from a plurality of computing devices. In some implementations, the PFT system computes and stores a total time for each activity for each patient and the wait time between adjacent activities for each patient.

In some implementations, for the plurality of activities, the PTF system displays user interfaces for indicating an actual start times and actual stop times on the plurality of computing device and receive from input devices at the computing devices the indications and send the actual start times and actual stop times over a network and store the actual start times and actual stop times in data storage.

In some implementations, the PTF system transmits information to a second computing device to render a display wherein the information transmitted comprises information for a plurality of scheduled activities. In some implementations, the information transmitted for each scheduled activity comprises a scheduled start time, a scheduled stop time, an actual start time, and an actual stop time. In some implementations, the information is transmitted to the second computing device as the information is received and the display is updated as the information is received.

In some implementations, the PTF system displays the plurality of scheduled activities on a timeline wherein each scheduled activity is displayed along a timeline corresponding to the scheduled start time and scheduled stop time for the scheduled activity. In some implementations, for each scheduled activity displayed, the PTF system simultaneously displays on the timeline the actual start time and actual stop time for the scheduled activity. In some implementations, the indicia for the scheduled activity is color-coded based on whether the scheduled activity is on-time or delayed.

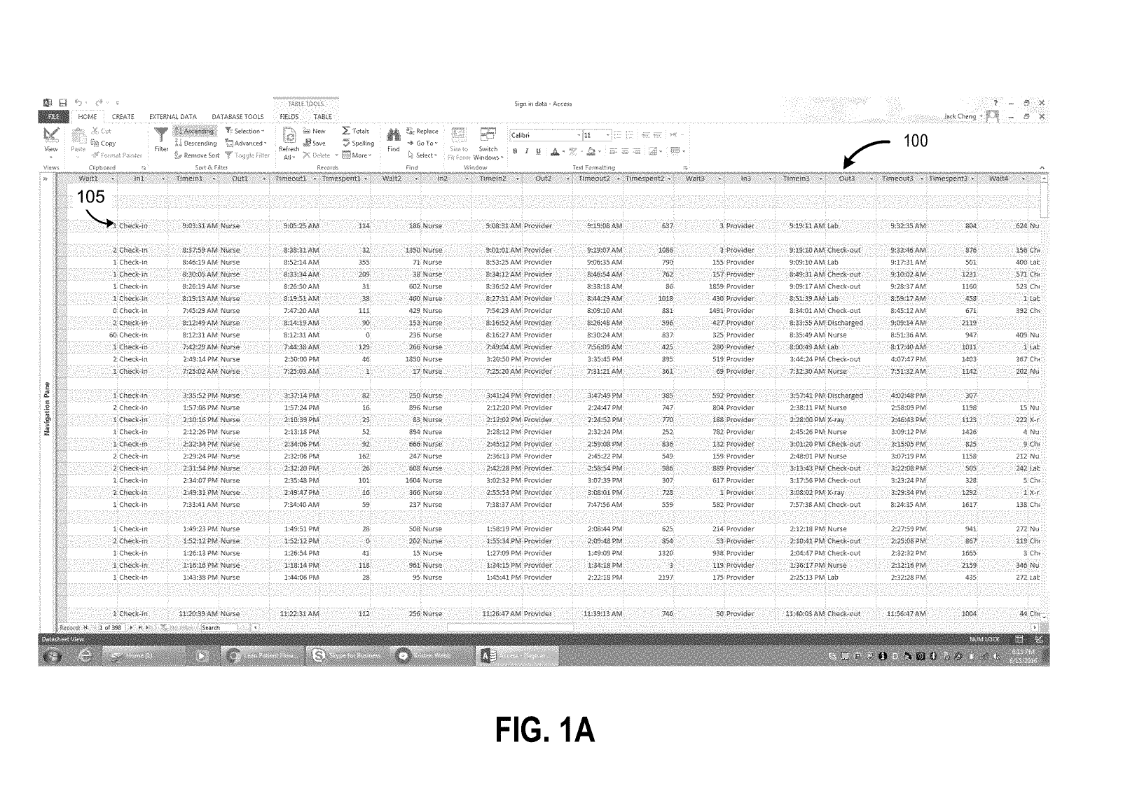

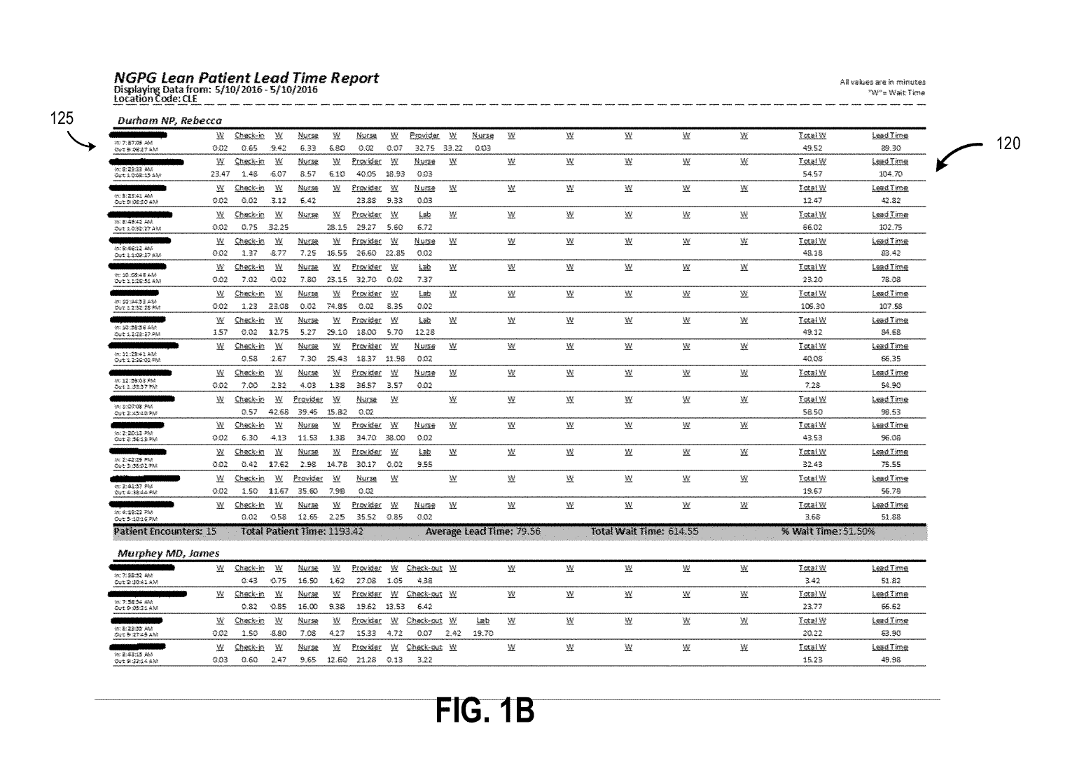

In some implementations, the PFT system is configured to capture data points (e.g., start, stop, and wait times from check-in to check out) for a patient based on actual office personnel interactions with the patient. FIG. 1A illustrates example raw data 100 captured using the Patient Flow Timer (PFT) system according to an implementation of the present disclosure. Thus, unlike existing system, the present disclosure is activity-based.

Referring to FIG. 1A, each row (e.g., row 105) in FIG. 1A represents a patient flow. As a patient moves through the medical office, data is recorded using the PFT system according to an implementation of the present disclosure. For example, referring to row 105, the start time (i.e., Timein1) and stop time (i.e., Timeout1) of Check-In is recorded and the total time spent during Check-In is calculated (i.e., Timespent1) as the difference between the stop time and start time. The activity, office person to be seen, or event after Check-In is recorded as “Out1”. As shown in row 105, for this particular patient flow, the next office person to be seen after Check-In is “Nurse.” Once the Nurse has recorded the start time (i.e., Timein2), the wait time from Check-In to Nurse can be calculated (i.e., Wait 2). Once the Nurse is finished with the patient, the stop time (i.e., Timeout2) is recorded and the total time spent with the Nurse is calculated (i.e., Timespent2) as the difference between the stop time and start time with the Nurse. The above process may be repeated for each activity, office person to be seen, or event that the patient will encounter during the patient's office visit. Thus, for each activity, office person to be seen, or event that the patient will encounter during the patient's office visit, a start time, stop time, and total time spent will be recorded. Furthermore, the wait time between adjacent activities, office persons, or events, will be calculated and recorded.

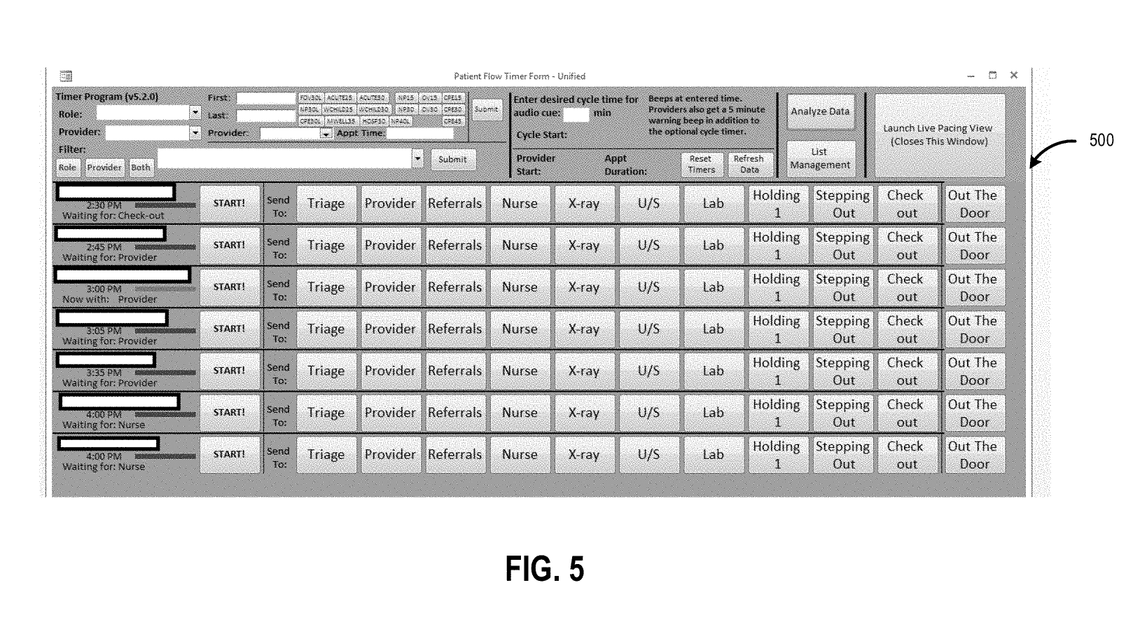

In some implementations, data may be recorded in real-time. That is, using the PFT system according to an implementation of the present disclosure, an office person may record a start time and a stop time simultaneously or within a predefined time of an event. As discussed below with reference to FIG. 5, the data may be captured using a computing device having a user-interface for capturing start times and stop times. In some implementations, the start times and stop times are captured when an office person interacts with the user-interface (e.g., uses an input device to interact with a designated area of the user interface). In some implementations, the computing device automatically captures a time when the office person interacts with the user-interface. As discussed in more detailed below, by capturing data real-time, real-time displays can be created to show the state of patient flow in an office and office wait times can be computed.

Once data is captured by the PFT system, data analysis can be performed on the data gathered. Reports generated from the data gathered may allow an office to evaluate the baseline functionality of aspects of patient flow in the office. For example, based on the data gathered using the PFT system according to an implementation of the present disclosure, it may be possible to review how many seconds it takes to check a patient in, how long a wait time for the radiology technician, how far behind a physician is running, etc.

FIG. 1B illustrates a sample report 120 that may be generated from the data gathered and/or calculated using the PFT system according to an implementation of the present disclosure. Each row represents a patient and the office personnel interaction times (time at Check-In, time with Nurse, time with Provider, wait times, etc). Each row shows the elapsed time for each activity that is indicated in the column for the particular patient. Each row also shows the total wait time as the sum of all the wait times (i.e., Total W) and the total time (i.e., Lead Time) a patient was in the office from Check-In (“In”) to Check-Out (“Out”).

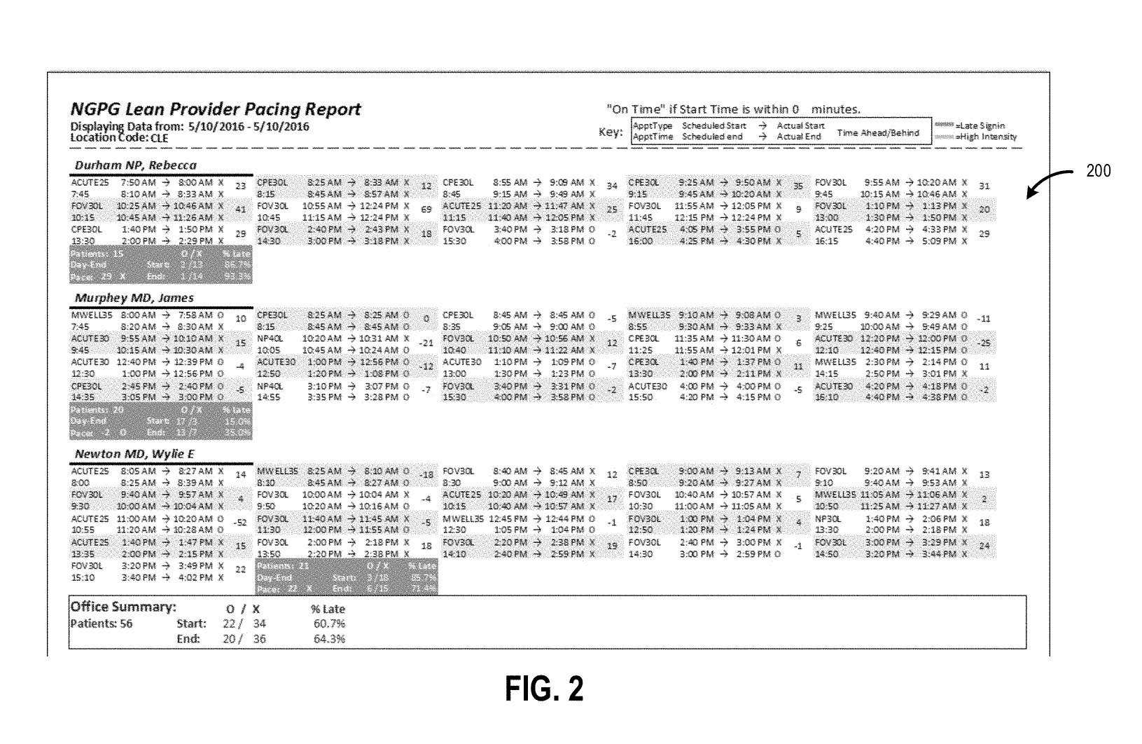

FIG. 2 illustrates another sample report 200 that may be generated from the data gathered and/or calculated using the PFT system according to an implementation of the present disclosure. FIG. 2 illustrates how often a provider (e.g., a doctor) starts and finishes a patient encounter “on time” based on the actual appointment times.

In some implementations, a patient encounter may be “on time” if the encounter began or ended within a predetermined time of the actual appointment time start or end, respectively. In some implementations, the predetermined time may be within the minute of the actual appointment time. Thus, if an appointment is scheduled at 3:00 PM and the provider starts at 3:01, the patient encounter may be considered not on-time or “late”. In some implementations, the predetermined time may be within the second of the actual appointment time. In some implementations, the predetermined time may be smaller or larger than one minute. In some implementations, the predetermined time may be larger than one second.

As shown in FIG. 2, the report 200 shows a tally of the number of late starts and late finishes for appointment within a preselected time period (e.g., day, week, etc.) per provider and for the office. For example, for Dr. Durham, FIG. 2 illustrates that out of 15 appointments/patient encounters on a particular day, Dr. Durham started 2 appointments on-time, started 13 appointments late, finished 1 appointment on time, and finished 14 appointments late. FIG. 2 also illustrates that 86.7% appointments started on time and 93.3% appointments ended late.

Still further, FIG. 2 illustrates the “PACE” for a provider (i.e., the time the provider ended his/her last scheduled appointment for the day compared to the scheduled end time for the last appointment for the day). If the provider finished his/her last scheduled appointment sooner than the scheduled end time for the last appointment for the day, then the provider's “PACE” value will be negative for the day. If the provider finished his/her last scheduled appointment later than the scheduled end time for the last appointment for the day, then the provider's “PACE” value will be positive for the day. If the provider finished his/her last scheduled appointment at the time of the scheduled end time for the last appointment for the day, then the provider's “PACE” value will be zero.

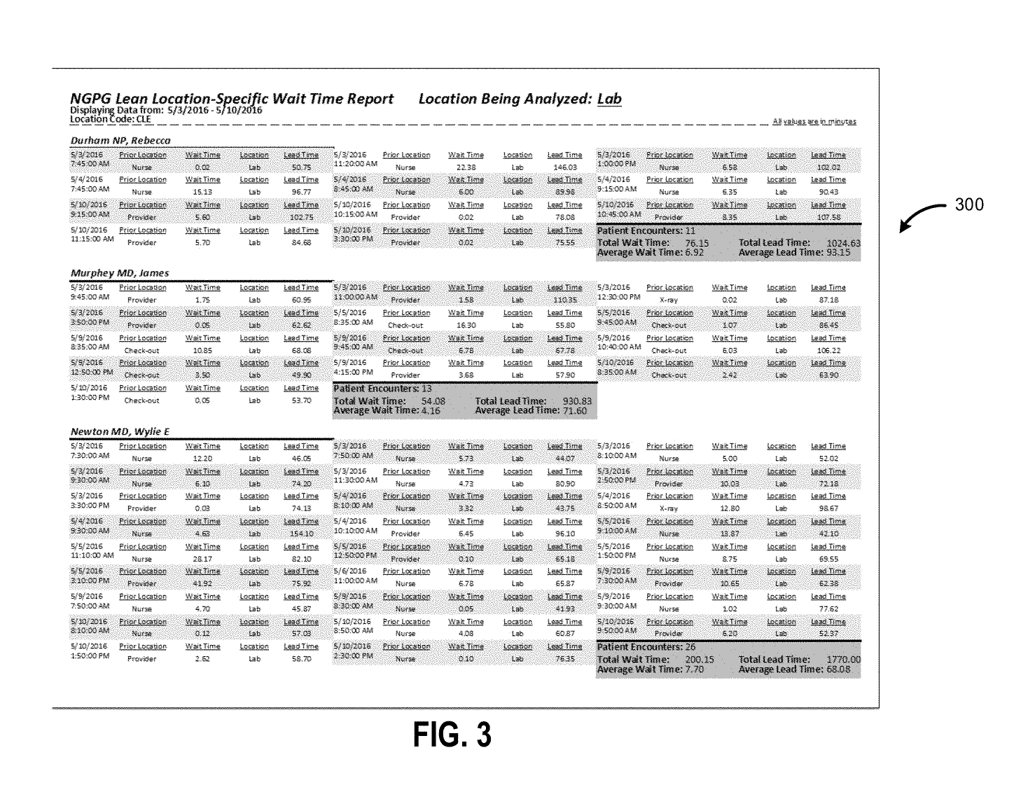

FIG. 3 illustrates another sample report 300 that may be generated from the data gathered and/or calculated using the PFT system according to an implementation of the present disclosure. FIG. 3 analyzes a specific activity (e.g., Lab) to determine if there is a significant wait time for that activity.

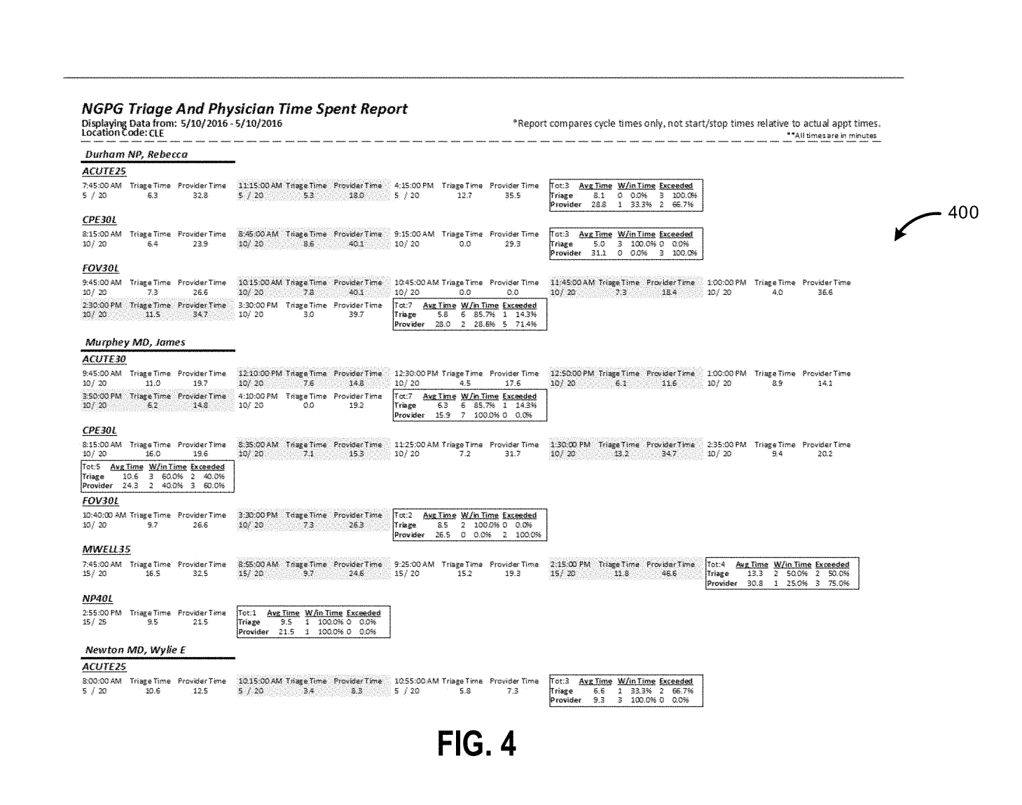

FIG. 4 illustrates yet another sample report 400 that may be generated from the data gathered using the PFT system according to an implementation of the present disclosure. FIG. 4 illustrates examples when a patient may sit in the same location (e.g., in an exam room) but two separate activities may occur there. FIG. 4 illustrates the actual interaction time of a nurse and a physician with a patient, grouped by appointment types (e.g., ACUTE25, CPE30L, FOV30L). In some implementations, some appointments are scheduled for a longer time than other appointments. For example, ACUTE25 is scheduled for 25 minutes while CPE30L and FOV30L are scheduled for 30 minutes.

As shown in FIG. 4, the report 400 shows for each activity within a preselected time period, the total time spent on the activity by a nurse (shown as “Triage Time”) and a physician (shown as “Provide Time”). The report 400 shows the scheduled time for the nurse and the physician for each active. For example, for D. Durham, the report shows for the ACUTE25 scheduled for 7:45 am, the nurse is scheduled to spend 5 minutes (i.e., cycle time) with the patient and the physician is scheduled to spend 20 minutes (i.e., cycle time) with the patients (indicated by the “5/20” designation under the 7:45:00 AM time).

The report 400 then shows a tally of the number of activities for the activity-type that were within the cycle time for the nurse and physical and the number of appointments for the activity-type that exceeded the cycle time for the nurse and physical. This information is also shown as a percentage. For example, FIG. 4 shows that for the three (3) ACUTE25 scheduled during the selected time period for Dr. Durham, the nurse completed 0 activities within the cycle time and exceeded the cycle time for 3 activities; the physician completed 1 activity within the cycle time and exceeded the cycle time for 2 activities.

FIG. 4 also shows the average time for the nurse and the physician for an activity-type for the selected time period.

With the information gathered using the PFT system according to an implementation of the present disclosure, a real-time view of the provider's (e.g., doctor) schedule with easy to understand visual color cues can quickly show if the clinical flow (e.g., schedule appointments) is on time or delayed.

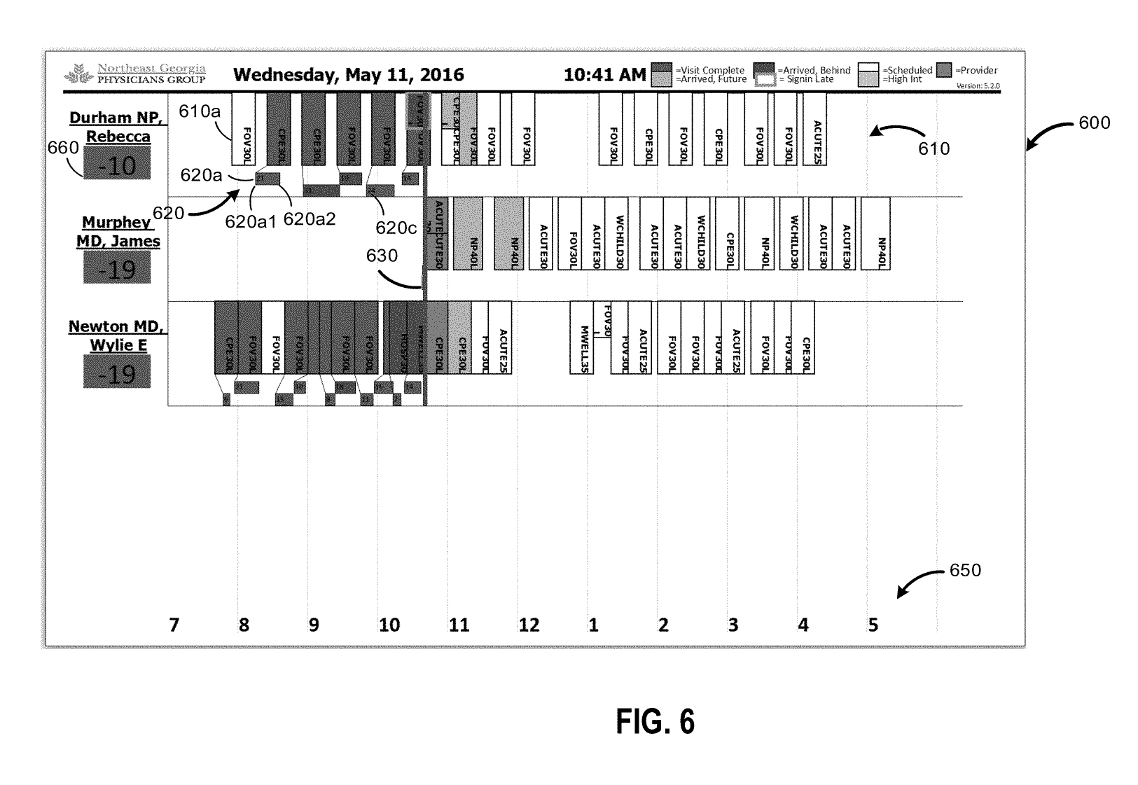

FIG. 6 illustrates an example real time display 600 of a physician's schedule 610 along a timeline 650 with the actual progression 620 of the schedule below based on data gathered using the PFT system according to an implementation of the present disclosure. The vertical bar 630 indicates the current time and moves along the timeline 650 based on the current time.

In some implementations, a provider's name 605 may be displayed on the leftmost side of the display and the scheduled activity 610 and actual schedule 620 for the provider may be to the right of the provider's name. In some implementations, the provider's name is displayed adjacent to the scheduled activity 610 and actual schedule 620 for the provider.

In some implementations, a plurality of providers and their scheduled activity and actual schedule may be shown on display 600.

In some implementations, each scheduled activity 610a is illustrated as a rectangle along a timeline corresponding to the actual scheduled time for the activity. In some implementations, the left side of the rectangle 610a1 corresponds to the scheduled start time and the right side of the rectangle corresponds to the scheduled stop time 610a2. In some implementations, each scheduled activity may be illustrated on the timeline in any suitable shape or manner.

In some implementations, the display 600 may indicate for each scheduled activity 610a, the physician's actual time for the activity 620a. In some implementations, the physician's actual time for a scheduled activity may be below and adjacent to the scheduled activity.

In some implementations, the physician's actual time for a scheduled activity (e.g., 620a) is illustrated as a rectangle on a timeline corresponding to the actual time for the activity. In some implementations, the left side of the rectangle 620a1 corresponds to the actual start time and the right side of the rectangle 620a2 corresponds to the actual stop time. In some implementations, the length of time for the activity 620c maybe indicated inside the rectangle. In some implementations, the physician's actual time for a scheduled activity may be illustrated on the timeline in any suitable shape or manner.

In some implementations, the rectangles may be color coded based on the current time and status of the scheduled activity. For example, if a patient has arrived early and checked-in, the rectangle corresponding to that scheduled activity may be a first color. A rectangle corresponding to a scheduled appointment that is complete may be a second color. A rectangle corresponding to a scheduled appointment where the patient has checked-in but the provider is late may be a third color. A rectangle corresponding to a scheduled appointment where the patient has not checked-in and is late may be a fourth color.

In some implementations, the display 600 may indicate the current state of the provider's schedule with a simple visual indicator (e.g., 660) such as a color or a number or both. In some implementations, the visual indicator may be below and adjacent to the provider's name. For example, in some implementations, the display 600 may indicate whether a provider is ahead of schedule by a first color (e.g., green) or behind schedule by a second color (e.g., red). In some implementations, the display 600 may indicate the amount of time a provider is ahead of schedule or behind schedule. The display 600 may indicate that a provider is ahead of schedule using a negative number and indicated that the provider is behind schedule using a positive number where the value is based on where the provider is and where the provider is supposed to be.

In some implementations, the state of the provider's schedule, the actual schedule 620, and/or the color codes, maybe updated in real-time based on data gathered using the PFT system according to an implementation of the present disclosure.

Based on the data gathered using the PFT system according to an implementation of the present disclosure, extra resources can be diverted in real-time to the provider team that is running behind. In some implementations, the PFT system includes a patient waiting room estimated wait time visual board for improved patient satisfaction and clarity. Implementations of a PFT system according to the present disclosure may provide a reliable objective dataset for continuous improvement of a medical office based on Lean principles.

In some implementations, each office personnel that interacts with a patient may have access to a computing device configured to a run a program to implement the PFT system according to an implementation of the present disclosure.

FIG. 5 illustrates an example user interface to allow office personnel to easily record interaction times with a patient according to an implementation of the present disclosure. For example, office personnel would access the program and for a particular client, click certain buttons on the screen to record start and stop times and other activity. FIG. 1A is an example of the data captured as a result of the process described below.

Referring to FIG. 5, in some implementations, each office personnel may select from a drop down menu a role (e.g., Nurse, X-ray, U/S, Lab, etc.]) or select his/her name from the provider drop down menu.

Each row of the user interface in FIG. 5 may be for a particular patient. Each row may be populated based on the role and/or provider selected in the drop down menus.

In some implementations, when a person from office personnel initially interacts with a patient, the person may press the start button. In some implementations, the start button may be pressed at another pre-defined activity so long that it is consistent across office personnel.

In some implementations, when the office person has completed the activity, the office person selects a button to indicate where to send the patient next (e.g., Send to: Triage). In some implementations, when the “Send to” button is pressed, the system records the time as the stop time for current activity.

If the office person, for example, pressed “Triage” for a particular patient, when an office person assigned for that patient selects “Triage” under the role drop down number, that patient would appear on the Triage office person's screen. When the Triage office person initially interacts with the patient, the Triage office person would then press “Start”. When the Triage office person has completed the activity, the Triage office person would select a button to indicate where to send the patient next (e.g., Send to: Lab).

If the Triage office person, for example, pressed “Lab” for a particular patient, when an office person assigned for that patient selects “Lab” under the role drop down number, that patient would appear on the Lab office person's screen. When the Lab office person initially interacts with the patient, the Lab office person would then press “Start”. When the Lab office person has completed the activity, the Lab office person would select a button to indicate where to send the patient (e.g., Send to: Nurse) next.

This process may continue until the “Out the Door” is selected. In this way, wait times between activities can be measured.

Also, as shown in the top middle section of FIG. 5, in some implementations, a user may enter a cycle time. In some implementations, the cycle time may be a predetermined time. In some implementations, the cycle time may be determined as part of the Lean model principles. For example, the cycle time may be the time determined to be an optimal time to complete an activity. In some implementations, the system may provide an audio warnings (e.g., a beep) to help the user stay on time based on the cycle time. In some implementations, when the role is “provider”, the system may automatically provide a predetermine warning (e.g., a 5 minute warning) for an office visit.

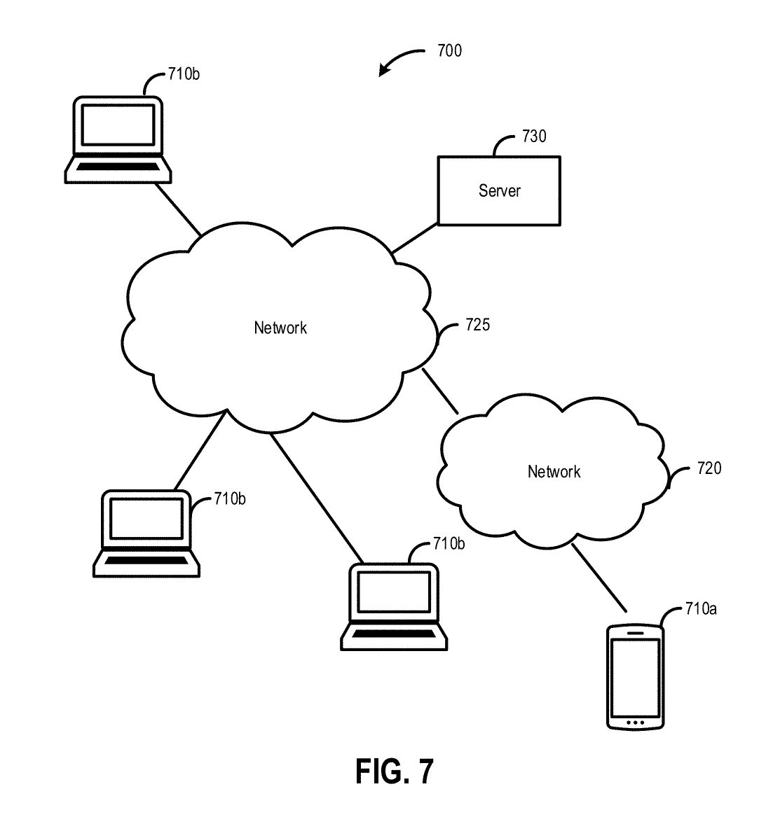

FIG. 7 illustrates an example environment 700 for recording interaction times with a patient according to an implementation of the present disclosure.

As shown in FIG. 7, the environment 700 can include client devices 710a and 710b (collectively client device 710), a wireless cellular network 720, a network 725, and a server 730. Client devices 710a and 710b are depicted as a mobile phone 710a and desktop computer 710b, respectively, but client devices 710 may comprise any type of computing device, such as a desktop computer system, a laptop, a smartphone, a mobile telephone, a tablet-style computer, any other handheld electronic device, or any other device capable of wireless or wired communication.

In some implementations, the client device 710a can connect to the network 725 through a wireless cellular network 720, such as GPRS-based and CDMA-based wireless networks, as well as 802.16 WiMax and long-range wireless data networks.

Client devices 710a and 710b can interact with the server 730 via an application, such as a web browser or a native application, residing on the client devices 710a and 710b to access the PFT system described above.

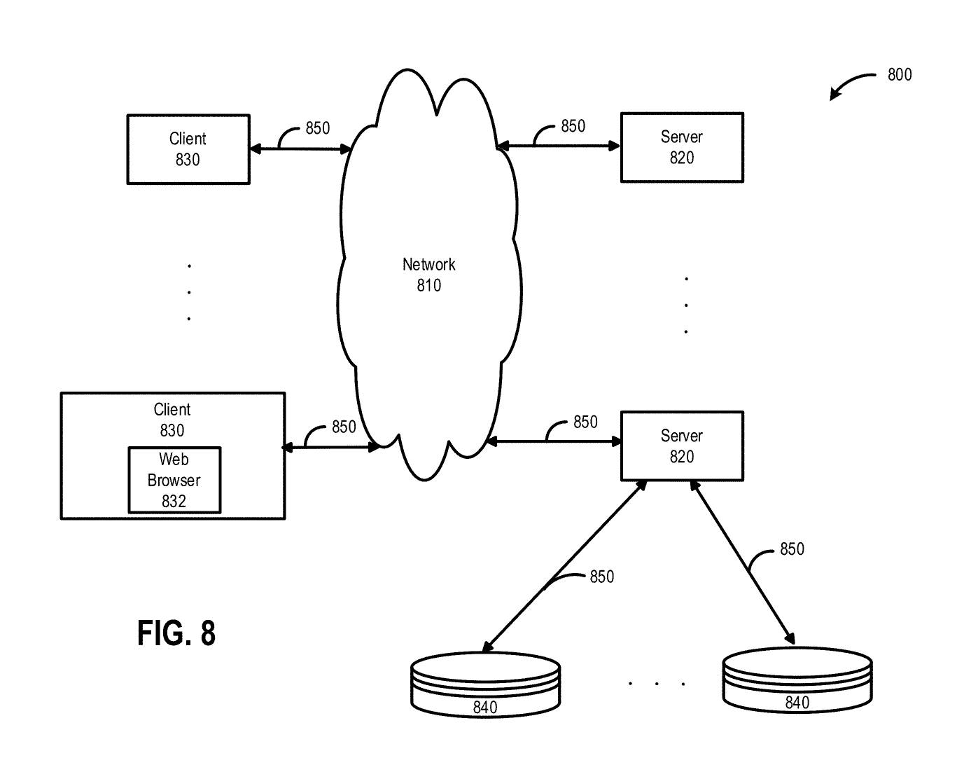

FIG. 8 illustrates another example environment 800 for recording interaction times with a patient using the PFT system according to an implementation of the present disclosure.

The example environment 800 can include a network 810, one or more servers 820, and one or more clients 830. The example environment 800 also can include one or more data storage 840 linked to one or more servers 420. Particular embodiments may be implemented in network environment 800. For example, applications used to render the user interfaces to perform the functions described above (e.g., recording patient interaction times, creating reports, and creating displays) may be written in software programs hosted by one or more servers 820.

In some implementations, network 810 may be an intranet, an extranet, a virtual private network (VPN), a local area network (LAN), a wireless LAN (WLAN), a wide area network (WAN), a metropolitan area network (MAN), a portion of the Internet, or another network 810 or a combination of two or more such networks 810. The present disclosure contemplates any suitable network 810.

One or more links 850 couple a server 820 or a client 830 to network 810. In some implementations, one or more links 850 each can include one or more wired, wireless, or optical links 850. In some implementations, one or more links 850 each can include an intranet, an extranet, a VPN, a LAN, a WLAN, a WAN, a MAN, a portion of the Internet, or another link 850 or a combination of two or more such links 850. The present disclosure contemplates any suitable links 850 coupling servers 820 and clients 830 to network 810.

In some implementations, each server 820 may be a unitary server or may be a distributed server spanning multiple computers or multiple datacenters. Servers 820 may be of various types, such as, for example and without limitation, web server, file server, application server, exchange server, database server, or proxy server. In some implementations, each server 820 may include hardware, software, or embedded logic components or a combination of two or more such components for carrying out the appropriate functionalities implemented or supported by server 820. For example, a web server is generally capable of hosting websites containing web pages or particular elements of web pages. More specifically, a web server may host HTML files or other file types, or may dynamically create or constitute files upon a request, and communicate them to clients 830 in response to HTTP or other requests from clients 830. A database server is generally capable of providing an interface for managing data stored in one or more data stores.

In some implementations, one or more data storages 840 may be communicatively linked to one or more servers 820 via one or more links 850. In some implementations, data storages 840 may be used to store various types of information. In some implementations, the information stored in data storages 840 may be organized according to specific data structures. In particular embodiment, each data storage 840 may be a relational database. Particular embodiments may provide interfaces that enable servers 820 or clients 830 to manage, e.g., retrieve, modify, add, or delete, the information stored in data storage 840.

In some implementations, each client 830 may be an electronic device including hardware, software, or embedded logic components or a combination of two or more such components and capable of carrying out the appropriate functions implemented or supported by client 830. For example and without limitation, a client 830 may be any type of computing device such as a desktop computer system, a laptop, a smartphone, a mobile telephone, a tablet-style computer, any other handheld electronic device, or any other device capable of wireless or wired communication. The present disclosure contemplates any suitable clients 830. A client 830 may enable a network user at client 830 to access network 830. A client 830 may enable its user to communicate with other users at other clients 830.

In some implementations, a client 830 may have a web browser 832, such as MICROSOFT INTERNET EXPLORER, GOOGLE CHROME, MOZILLA FIREFOX, or any other future developed web browser and may have one or more add-ons, plug-ins, or other extensions. A user at client 830 may enter a Uniform Resource Locator (URL) or other address directing the web browser 832 to a server 820, and the web browser 832 may generate a Hyper Text Transfer Protocol (HTTP) request and communicate the HTTP request to server 820. Server 820 may accept the HTTP request and communicate to client 830 one or more Hyper Text Markup Language (HTML) files responsive to the HTTP request. Client 830 may render a web page based on the HTML files from server 820 for presentation to the user. The present disclosure contemplates any suitable web page files. As an example and not by way of limitation, web pages may render from HTML files, Extensible Hyper Text Markup Language (XHTML) files, or Extensible Markup Language (XML) files, according to particular needs. Such pages may also execute scripts such as, for example and without limitation, those written in JAVASCRIPT, JAVA, MICROSOFT SILVERLIGHT, combinations of markup language and scripts such as AJAX (Asynchronous JAVASCRIPT and XML), and the like. Herein, reference to a web page encompasses one or more corresponding web page files (which a browser may use to render the web page) and vice versa, where appropriate.

In some implementations, a client 830 having a web browser 832 can connect to a server 820 to use the PTF system for recording patient interaction times. In some implementations, input received from an user may be stored in a data storage 840 communicatively linked to the servers 420.

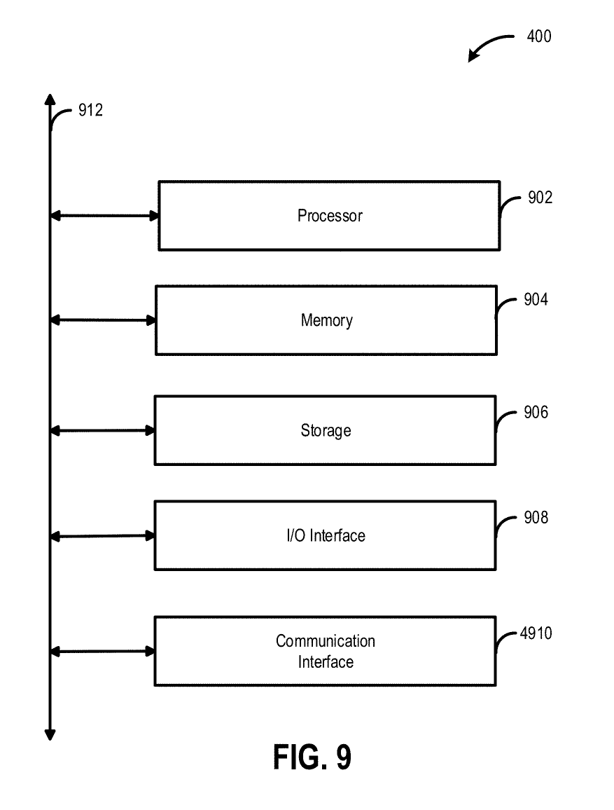

FIG. 9 illustrates an example computer system 900, which may be used with some embodiments of the present invention. This disclosure contemplates any suitable number of computer systems 900. This disclosure contemplates computer system 900 taking any suitable physical form. As example and not by way of limitation, computer system 900 may be an embedded computer system, a system-on-chip (SOC), a single-board computer system (SBC) (such as, for example, a computer-on-module (COM) or system-on-module (SOM)), a desktop computer system, a laptop, an interactive kiosk, a mainframe, a mesh of computer systems, a mobile telephone, a personal digital assistant (PDA), a server, or a combination of two or more of these. Where appropriate, computer system 900 may include one or more computer systems 900; be unitary or distributed; span multiple locations; span multiple machines; or reside in a cloud, which may include one or more cloud components in one or more networks. Where appropriate, one or more computer systems 900 may perform without substantial spatial or temporal limitation one or more steps of one or more methods described or illustrated herein. As an example and not by way of limitation, one or more computer systems 900 may perform in real time or in batch mode one or more steps of one or more methods described or illustrated herein. One or more computer systems 900 may perform at different times or at different locations one or more steps of one or more methods described or illustrated herein, where appropriate.

In some implementations, computer system 900 includes a processor 902, memory 904, storage 906, an input/output (I/O) interface 908, a communication interface 910, and a bus 912. Although this disclosure describes and illustrates a particular computer system having a particular number of particular components in a particular arrangement, this disclosure contemplates any suitable computer system having any suitable number of any suitable components in any suitable arrangement.

In some implementations, processor 902 includes hardware for executing instructions, such as those making up a computer program. As an example and not by way of limitation, to execute instructions, processor 902 may retrieve (or fetch) the instructions from an internal register, an internal cache, memory 904, or storage 906; decode and execute them; and then write one or more results to an internal register, an internal cache, memory 904, or storage 906. In some implementations, processor 902 may include one or more internal caches for data, instructions, or addresses. The present disclosure contemplates processor 902 including any suitable number of any suitable internal caches, where appropriate. As an example and not by way of limitation, processor 902 may include one or more instruction caches, one or more data caches, and one or more translation look-aside buffers (TLBs). Instructions in the instruction caches may be copies of instructions in memory 904 or storage 906, and the instruction caches may speed up retrieval of those instructions by processor 902. Data in the data caches may be copies of data in memory 904 or storage 906 for instructions executing at processor 902 to operate on; the results of previous instructions executed at processor 902 for access by subsequent instructions executing at processor 902 or for writing to memory 904 or storage 906; or other suitable data. The data caches may speed up read or write operations by processor 902. The TLBs may speed up virtual-address translation for processor 902. In some implementations, processor 902 may include one or more internal registers for data, instructions, or addresses. The present disclosure contemplates processor 902 including any suitable number of any suitable internal registers, where appropriate. Where appropriate, processor 902 may include one or more arithmetic logic units (ALUs); be a multi-core processor; or include one or more processors 902. Although this disclosure describes and illustrates a particular processor, this disclosure contemplates any suitable processor.

In some implementations, memory 904 includes main memory for storing instructions for processor 902 to execute or data for processor 902 to operate on. As an example and not by way of limitation, computer system 900 may load instructions from storage 906 or another source (such as, for example, another computer system 900) to memory 904. Processor 902 may then load the instructions from memory 904 to an internal register or internal cache. To execute the instructions, processor 902 may retrieve the instructions from the internal register or internal cache and decode them. During or after execution of the instructions, processor 902 may write one or more results (which may be intermediate or final results) to the internal register or internal cache. Processor 902 may then write one or more of those results to memory 904. In some implementations, processor 402 executes only instructions in one or more internal registers or internal caches or in memory 904 (as opposed to storage 906 or elsewhere) and operates only on data in one or more internal registers or internal caches or in memory 904 (as opposed to storage 906 or elsewhere). One or more memory buses (which may each include an address bus and a data bus) may couple processor 402 to memory 904. Bus 912 may include one or more memory buses, as described below. In some implementations, one or more memory management units (MMUs) reside between processor 902 and memory 904 and facilitate accesses to memory 904 requested by processor 902. In some implementations, memory 904 includes random access memory (RAM). This RAM may be volatile memory, where appropriate Where appropriate, this RAM may be dynamic RAM (DRAM) or static RAM (SRAM). Moreover, where appropriate, this RAM may be single-ported or multi-ported RAM. The present disclosure contemplates any suitable RAM. Memory 904 may include one or more memories 902, where appropriate. Although this disclosure describes and illustrates particular memory, this disclosure contemplates any suitable memory.

In some implementations, storage 906 includes mass storage for data or instructions. As an example and not by way of limitation, storage 906 may include an HDD, a floppy disk drive, flash memory, an optical disc, a magneto-optical disc, magnetic tape, or a Universal Serial Bus (USB) drive or a combination of two or more of these. Storage 906 may include removable or non-removable (or fixed) media, where appropriate. Storage 906 may be internal or external to computer system 900, where appropriate. In some implementations, storage 906 is non-volatile, solid-state memory. In some implementations, storage 906 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 906 taking any suitable physical form. Storage 906 may include one or more storage control units facilitating communication between processor 902 and storage 906, where appropriate. Where appropriate, storage 906 may include one or more storages 906. Although this disclosure describes and illustrates particular storage, this disclosure contemplates any suitable storage.

In some implementations, I/O interface 908 includes hardware, software, or both providing one or more interfaces for communication between computer system 900 and one or more I/O devices. Computer system 900 may include one or more of these I/O devices, where appropriate. One or more of these I/O devices may enable communication between a person and computer system 900. As an example and not by way of limitation, an I/O device may include a keyboard, keypad, microphone, monitor, mouse, printer, scanner, speaker, still camera, stylus, tablet, touch screen, trackball, video camera, another suitable I/O device or a combination of two or more of these. An I/O device may include one or more sensors. This disclosure contemplates any suitable I/O devices and any suitable I/O interfaces 908 for them. Where appropriate, I/O interface 908 may include one or more device or software drivers enabling processor 902 to drive one or more of these I/O devices. I/O interface 908 may include one or more I/O interfaces 908, where appropriate. Although this disclosure describes and illustrates a particular I/O interface, this disclosure contemplates any suitable I/O interface.

In some implementations, communication interface 910 includes hardware, software, or both providing one or more interfaces for communication (such as, for example, packet-based communication) between computer system 900 and one or more other computer systems 900 or one or more networks. As an example and not by way of limitation, communication interface 910 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 910 for it. As an example and not by way of limitation, computer system 900 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 900 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. Computer system 900 may include any suitable communication interface 910 for any of these networks, where appropriate. Communication interface 910 may include one or more communication interfaces 910, where appropriate. Although this disclosure describes and illustrates a particular communication interface, this disclosure contemplates any suitable communication interface.

In some implementations, bus 912 includes hardware, software, or both coupling components of computer system 900 to each other. As an example and not by way of limitation, bus 912 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCI-X) bus, a serial advanced technology attachment (SATA) bus, a Video Electronics Standards Association local (VLB) bus, or another suitable bus or a combination of two or more of these. Bus 912 may include one or more buses 912, where appropriate. Although this disclosure describes and illustrates a particular bus, this disclosure contemplates any suitable bus or interconnect.

Herein, reference to a computer-readable storage medium encompasses one or more non-transitory, tangible computer-readable storage media possessing structure. As an example and not by way of limitation, a computer-readable storage medium may include a semiconductor-based or other integrated circuit (IC) (such, as for example, a field-programmable gate array (FPGA) or an application-specific IC (ASIC)), a hard disk, an HDD, a hybrid hard drive (HHD), an optical disc, an optical disc drive (ODD), a magneto-optical disc, a magneto-optical drive, a floppy disk, a floppy disk drive (FDD), magnetic tape, a holographic storage medium, a solid-state drive (SSD), a RAM-drive, a SECURE DIGITAL card, a SECURE DIGITAL drive, or another suitable computer-readable storage medium or a combination of two or more of these, where appropriate. Herein, reference to a computer-readable storage medium excludes any medium that is not eligible for patent protection under 35 U.S.C. § 101. Herein, reference to a computer-readable storage medium excludes transitory forms of signal transmission (such as a propagating electrical or electromagnetic signal per se) to the extent that they are not eligible for patent protection under 35 U.S.C. § 101.

This disclosure contemplates one or more computer-readable storage media implementing any suitable storage. In some implementations, a computer-readable storage medium implements one or more portions of processor 902 (such as, for example, one or more internal registers or caches), one or more portions of memory 904, one or more portions of storage 906, or a combination of these, where appropriate. In some implementations, a computer-readable storage medium implements RAM or ROM. In some implementations, a computer-readable storage medium implements volatile or persistent memory. In some implementations, one or more computer-readable storage media embody software. Herein, reference to software may encompass one or more applications, bytecode, one or more computer programs, one or more executables, one or more instructions, logic, machine code, one or more scripts, or source code, and vice versa, where appropriate. In some implementations, software includes one or more application programming interfaces (APIs). This disclosure contemplates any suitable software written or otherwise expressed in any suitable programming language or combination of programming languages. In some implementations, software is expressed as source code or object code. In some implementations, software is expressed in a higher-level programming language, such as, for example, C, Perl, or a suitable extension thereof. In some implementations, software is expressed in a lower-level programming language, such as assembly language (or machine code). In some implementations, software is expressed in JAVA. In some implementations, software is expressed in Hyper Text Markup Language (HTML), Extensible Markup Language (XML), or other suitable markup language.

The foregoing description of the embodiments of the invention has been presented for the purpose of illustration; it is not intended to be exhaustive or to limit the invention to the precise forms disclosed. Persons skilled in the relevant art can appreciate that many modifications and variations are possible in light of the above disclosure. For example. it will apparent to one of ordinary skill in the art that the invention may be used with any electronic network service, even if it is not provided through a website. Any computer-based system that provides networking functionality can be used in accordance with the present invention even if it relies, for example, on email, instant messaging or other forms of peer-to-peer communications, and any other technique for communicating between users. The invention is thus not limited to any particular type of communication system, network, protocol, format or application.

Some portions of this description describe the embodiments of the invention in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof

Any of the steps, operations, or processes described herein may be performed or implemented with one or more hardware or software modules, alone or in combination with other devices. In one embodiment, a software module is implemented with a computer program product comprising a computer-readable medium containing computer program code, which can be executed by a computer processor for performing any or all of the steps, operations, or processes described.

Embodiments of the invention may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a tangible computer readable storage medium or any type of media suitable for storing electronic instructions, and coupled to a computer system bus. Furthermore, any computing systems referred to in the specification may include a single processor or may be architectures employing multiple processor designs for increased computing capability.

While the foregoing processes and mechanisms can be implemented by a wide variety of physical systems and in a wide variety of network and computing environments, the server or computing systems described below provide example computing system architectures for didactic, rather than limiting, purposes.

The present invention has been explained with reference to specific embodiments. For example, while embodiments of the present invention have been described as operating in connection with a network system, the present invention can be used in connection with any communications facility that allows for communication of messages between users, such as an email hosting site. Other embodiments will be evident to those of ordinary skill in the art. It is therefore not intended that the present invention be limited, except as indicated by the appended claims.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention, which is set forth in the following claims.

The present disclosure encompasses all changes, substitutions, variations, alterations, and modifications to the example embodiments herein that a person having ordinary skill in the art would comprehend.

1. A computer-implemented method for measuring patients' movement and wait times through a medical office, the method comprising:
for a plurality of activities at a medical office for a patient as the patient moves through a medical office for services, receiving for each activity for each patient at a first computing device an actual start time of the activity and an actual stop time of the activity, wherein the actual start times and actual stop times are transmitted over a network from a plurality of computing devices;
computing and storing a total time for each activity for each patient and the wait time between adjacent activities for each patient;
for the plurality of activities, displaying user interfaces for indicating an actual start times and actual stop times on the plurality of computing device and receiving from input devices at the computing devices the indications and sending the actual start times and actual stop times over a network and storing the actual start times and actual stop times in data storage;
transmitting information to a second computing device to render a display wherein the information transmitted comprises information for a plurality of scheduled activities, where the information transmitted for each scheduled activity comprises a scheduled start time, a scheduled stop time, an actual start time, and an actual stop time, wherein the information is transmitted to the second computing device as the information is received and the display is updated as the information is received; and
displaying the plurality of scheduled activities on a timeline wherein each scheduled activity is displayed along a timeline corresponding to the scheduled start time and scheduled stop time for the scheduled activity, and for each scheduled activity displayed, simultaneously displaying on the timeline the actual start time and actual stop time for the scheduled activity, wherein the indicia for the scheduled activity is color-coded based on whether the scheduled activity is on-time or delayed.

2. The computer-implemented method of claim 1, wherein the plurality of activities at least comprises check-in, check-out, time with a nurse, and time with a doctor.

3. The computer-implemented method of claim 2, wherein the plurality of activities further comprises at least one other activity involving an office person.

4. The computer-implemented method of claim 1, further comprising computing the total wait time the patient and the total time the patient was in the office.

5. The computer-implemented method of claim 1, further comprising receiving for each activity a role or name and computing a performance based on scheduled times and actual times for activities associated with a particular role or name.

6. The computer-implemented method of claim 1, further comprising comparing an actual end time for a last scheduled appointment for a day with a scheduled end time for the last scheduled appointment.

7. The computer-implemented method of claim 1, further comprising receiving for each activity a name and displaying the plurality of scheduled activities on the timeline grouped by name wherein the name is displayed on the leftmost side of the display and the scheduled activities associated with the name are displayed adjacent to the name and wherein the indicia for the actual times for each scheduled activity appears below and adjacent to the indicia for the scheduled times for each scheduled activity

8. The computer-implemented method of claim 7 wherein the indicia for the scheduled times for each scheduled activity and the indicia for the actual times for each scheduled activity is displayed as a rectangle on the timeline.

9. The computer-implemented method of claim 7, further comprising displaying simultaneously a visual indicator of the current state of a schedule associated with a name wherein the visual indicator indicates whether the actual schedule is ahead or behind the scheduled times.

10. The computer-implemented method of claim 1 wherein the user interfaces for indicating an actual start times and actual stop times on the plurality of computing device further display indicia for inputting a role or name, the method further comprising and displaying a plurality of rows on the user interfaces based on the role or name inputted at the input devices wherein each row corresponds to a patient assigned for that role or name, wherein each row comprises an indicia to indicate a start time for that patient and indicia to indicate the stop time for the patient.

11. The computer-implemented method of claim 10 wherein the indicia to indicate the stop time for the patient is an indicia for the next activity for the patient.

12. The computer-implemented method of claim 10 wherein the user interface indicates a time to complete an activity and the computing devices provides an audio warning at a predetermined time before a scheduled end time.
